SEQUENCE LISTING (24 sequences)

SEQ ID NO: 1 - protein, 9 aa, Artificial sequence
  Peptide derived from the defensin of Mytilus galloprovincialis
CGGWHRLRC

SEQ ID NO: 2 - protein, 9 aa, Artificial sequence
  Peptide derived from the defensin of Mytilus galloprovincialis
CGGWKRKRC

SEQ ID NO: 3 - protein, 39 aa, Mytilus galloprovincialis
GFGCPNNYQCHRHCKSIPGRCGGYCGGWHRLRCTCYRCG

SEQ ID NO: 4 - protein, 13 aa, Artificial sequence
  Peptide derived from the defensin of Mytilus galloprovincialis
SGGYCGGWHRLRC

SEQ ID NO: 5 - protein, 19 aa, Artificial sequence
  Peptide derived from the defensin of Mytilus galloprovincialis
CGGYSGGWHRLRSTSYRCG

SEQ ID NO: 6 - protein, 19 aa, Artificial sequence
  Peptide derived from the defensin of Mytilus galloprovincialis
SGGYCGGWHRLRCTSYRSG

SEQ ID NO: 7 - protein, 19 aa, Artificial sequence
  Peptide derived from the defensin of Mytilus galloprovincialis
CGGYCGGWHRLRCTSYRCG

SEQ ID NO: 8 - protein, 10 aa, Artificial sequence
  Peptide derived from the defensin of Mytilus galloprovincialis
GFGSPNNYQS

SEQ ID NO: 9 - protein, 20 aa, Artificial sequence
  Peptide derived from the defensin of Mytilus galloprovincialis
GFGSPNNYQSHRHSKSIPGR

SEQ ID NO: 10 - protein, 18 aa, Artificial sequence
  Peptide derived from the defensin of Mytilus galloprovincialis
PNNYQSHRHSKSIPGRGR

SEQ ID NO: 11 - protein, 17 aa, Artificial sequence
  Peptide derived from the defensin of Mytilus galloprovincialis
PNNYQSHRHCKSIPGRC

SEQ ID NO: 12 - protein, 17 aa, Artificial sequence
  Peptide derived from the defensin of Mytilus galloprovincialis
PNNYQSHRHSKSIPGRS

SEQ ID NO: 13 - protein, 10 aa, Artificial sequence
  Peptide derived from the defensin of Mytilus galloprovincialis
NYQSHRHSKS

SEQ ID NO: 14 - protein, 9 aa, Artificial sequence
  Peptide derived from the defensin of Mytilus galloprovincialis
SKSIPGRSG

SEQ ID NO: 15 - protein, 9 aa, Artificial sequence
  Peptide derived from the defensin of Mytilus galloprovincialis
CKSIPGRCG

SEQ ID NO: 16 - protein, 12 aa, Artificial sequence
  Peptide derived from the defensin of Mytilus galloprovincialis
CGGYSSTSYRCG

SEQ ID NO: 17 - protein, 9 aa, Artificial sequence
  Peptide derived from the defensin of Mytilus galloprovincialis
SGGWHRLRS

SEQ ID NO: 18 - protein, 10 aa, Artificial sequence
  Peptide derived from the defensin of Mytilus galloprovincialis
RLRSTSYRSG

SEQ ID NO: 19 - protein, 9 aa, Artificial sequence
  Peptide derived from the defensin of Mytilus galloprovincialis
CGGWKRLRC

SEQ ID NO: 20 - protein, 9 aa, Artificial sequence
  Peptide derived from the defensin of Mytilus galloprovincialis
CGGWKRKRC

SEQ ID NO: 21 - protein, 9 aa, Artificial sequence
  Peptide derived from the defensin of Mytilus galloprovincialis
CKKWKRKRC

SEQ ID NO: 22 - protein, 8 aa, Artificial sequence
  Peptide derived from the defensin of Mytilus galloprovincialis
CKWKRKRC

SEQ ID NO: 23 - protein, 7 aa, Artificial sequence
  Peptide derived from the defensin of Mytilus galloprovincialis
CWKRKRC

SEQ ID NO: 24 - protein, 6 aa, Artificial sequence
  Peptide derived from the defensin of Mytilus galloprovincialis
CWKRKC

(19) United States
(12) Patent Application Publication
(10) Pub. No.: US 2006/0241026 A1
(43) Pub. Date: Oct. 26, 2006

(54) Novel cyclic peptides and use thereof as anti-microbial agents

(76) Inventors: Bernard Romestand, Saint Gely Du Fesc (FR); Claude Granier, Clapier (FR); Philippe Roch, Montpellier (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/513,637

(22) PCT Filed: May 5, 2003
(86) PCT No.: PCT/FR03/01387

§ 371 Date: May 16, 2005

(30) Foreign Application Priority Data
May 7, 2002 (FR) 02/05704

(51) Int. Cl. A61K 38/12; C07K 7/64; C07K 7/08

(52) U.S. Cl. 514/9; 530/317; 530/329

(57) ABSTRACT

The invention relates to compounds selected from: peptides having formula (1): C^{(s)}—X₁—X₂—X₃—X₄—X₅—X₆—X₇-C^{(s)}, wherein the two cysteine residues are linked by means of a disulphide bridge which is represented by symbol C^{(s)} and X₁, X₂, X₃X₄, X₅, X₆ and X₇ denote amino acids selected from a determined list; and derivatives of said peptides. The invention also relates to compositions comprising same and to the use thereof as anti-microbial agents.

The present invention relates to novel anti-microbial agents, and more particularly to low molecular weight peptides derived from the defensin of the Mediterranean mussel or Mytilus galloprovincialis. It also relates to a method for preparing them and to their applications, in particular their use for preparing a medicinal product.

The discovery of antibiotics was without doubt one of the greatest medical achievements of the 20th century. Today, more than 150 antibiotics are marketed and tens of novel compounds are undergoing authorization. However, most antibiotics correspond to only about half a dozen different families and the novel molecules are in fact only variants of already known compounds (Breihaupt, 1999, Nature 17; 1165-9). These families of molecules no longer make it possible to neutralize the emergence of new mutant pathogens that are resistant to antibiotics. In the hospital environment, the situation is alarming: nosocomial infections can affect 10% of patients and can prove to be extremely formidable in patients suffering from the human immunodeficiency virus or HIV, or in individuals in a situation of immunosuppression, in particular following transplants or during anticancer treatments.

In aquaculture, zootechnical progress focused on increasing productions (bivalve mollusks and crustacea) results in the creation of conditions favorable to the appearance and to the development of bacterial, viral and parasitic diseases. In addition, the sometimes excessive increase in farming densities can affect the physiological and immune state of the animals, which then become more sensitive to pathogenic or opportunistic agents. The obtaining of better prophylaxis for individuals against these diseases is currently being sought.

The identification of new compounds, derived from plants and from animals, which may be anti-microbial agents makes it possible to envision the development of novel medicinal products. Cationic anti-microbial peptides in particular represent a very advantageous path of development.

Since the discovery of the 1st anti-microbial peptide, cecropin, in the lepidoptoran Hyalophora cecropia by the team of Hans Boman (Steiner et al., 1981, Nature 292, 246-248), more than 400 anti-microbial peptides have been identified from various organisms, including the mussel Mytilus edulis (Charlet et al, 1999, J. Biol. Chem. 271; 21808-13) and Mytilus galloprovincialis (Hubert et al, 1996, Eur. J. Biochem. 240; 302-6; Mitta et al, 1999, J. Cell. Sci., 112; 4233-42); these peptides with anti-microbial activity, which are homologs of arthropod defensins, have been described. Their originality lies in their mode of action since it is different from that of conventional antibiotics. Even though their mode of action has not been clearly elucidated, it is accepted that cationic anti-bacterial peptides kill microorganisms by disorganizing or perforating the cytoplasmic membranes (Maloy and Kari, 1995, Biopolymers 37:105-22; Falla et al, 1996, J. Biol. Chem. 271:19298-303; Epand and Vogel, 1999, Biochim. Biophys Acta 1462:11-28). Their specificity of action is based on the differences in composition and in physicochemical properties which differentiate microbial membranes from eukaryotic cell membranes (Friedrich et al, 2000, Antimicrob. Agents Chemoth. 44:2086-2092). It is a phenomenon which is generally rapid and irreversible and which, by its very nature, decreases the risk of the emergence of resistant individuals; this constitutes an important asset in the perspective of the development of novel antibiotics.

By virtue of document U.S. Pat. No. 5,821,224, cysteine-rich anti-microbial peptides derived from bovine neutrophils, called beta-defensins, are known. Among the peptides studied in that document, it appeared that those comprising the N-terminal end of the natural defensin exhibited the most advantageous anti-microbial activity profile.

Document WO 98/40091 describes anti-microbial peptides in which the sequence is derived from the amino acid sequences from natural defensins and bactenecins.

Document WO 01/09175 describes anti-microbial peptides derived from a plant defensin, in which the sequence is modified so as to be either enriched in cysteine residues, or lacking in residues capable of forming disulfide bridges.

Document WO 01/09174 describes anti-microbial peptides derived from plant defensins by substitution of one or more amino acids.

Document WO 00/68625 describes cyclic peptides, called theta-defensins, having anti-microbial activity. These peptides are derived from natural or modified linear peptides and peptides that are cyclized by formation of a peptide bond between the N-terminal amine and the C-terminal carbon. Peptides cyclized in this way are not laid open to the action of exdpeptidases and are therefore liable to exhibit a resistance to proteolysis that is greater than that of the natural peptides.

Novel classes of molecules capable of treating microbial infections have thus emerged. However, molecules capable of treating these infections are still sought, since resistances emerge as treatments are developed. In addition, greater effectiveness and selectivity with respect to the microorganisms targeted is always being sought. The aim of the present invention is to provide new molecules exhibiting an advantageous spectrum of anti-microbial activity and exhibiting unexpected properties with respect to those of the molecules of the prior art. In addition, the molecules according to the invention can be prepared easily at a cost which makes it possible to envision industrial and commercial production.

A subject of the invention is novel cyclic peptides, or peptides comprising a cyclic fragment, and also certain of their chemical derivatives, which exhibit anti-microbial, in particular anti-bacterial or anti-fungal, activity, these peptides comprising an unnatural bridge, preferably a disulfide bridge, these peptides being derived from Mytilus galloprovincialis defensin, referred to as MGD 1.

Mytilus galloprovincialis defensin, MGD 1, is known for its anti-microbial activity (Hubert et al., 1996, Eur. J. Biochem., 240:302-6; Mitta et al., 1999, J. Cell. Sci., 112, 4233-42).

However, the use of native MGD 1 as an anti-microbial agent comes up against a substantial difficulty: the extraction and purification thereof from the mussel are difficult to obtain with a satisfactory yield.

Moreover, the chemical synthesis is performed with an overall yield that is very poor because of the difficulty in forming the 4 disulfide bridges.

The first peptide according to the invention corresponds to the sequence SEQ ID NO:1 C^{(S)}GGWHRLRC^{(S)} in which the two cysteine residues are linked via a disulfide bridge.

A subject of the invention is also homologs of the peptide SEQ ID NO:1 and some chemical derivatives of this peptide. For the purpose of the present invention, the term “homologs” is intended to mean peptides in which the amino acid sequence exhibits at least 60% similarity with the sequence SEQ ID NO:1, even more preferably 70%, yet even more preferably 80%, preferably at least 90%, and even more favorably at least 95%, or better still 98%, similarity with the sequence SEQ ID NO:1, it being understood that the sequences concerned comprise a cysteine residue at each of their ends, these terminal cysteine residues being linked via a disulfide bridge.

The expression “X % similarity between the peptide P and the sequence SEQ ID NO:1” is intended to mean that, when the sequence P is aligned opposite SEQ ID NO:1, in the same direction, X % of the amino acids of P are identical to the corresponding amino acid of SEQ ID NO:1 or are replaced with an amino acid of the same class, it being understood that, if the sequences are not the same length, a space will be placed between the amino acids of the sequence concerned. The degree of homology can be evaluated by methods well known to those skilled in the art (for example, WILBUR W. J. et al Proceedings of the National Academy of Sciences USA 80, 726-730 (1983); MYERS et al, Comput. Appl. Biosci. 4, 11-17 (1988)).

For the purpose of the present invention, the homology with SEQ ID NO:1 extends to the peptides comprising from 6 to 15 amino acids and the sequence of which, once aligned with SEQ ID NO:1, with the spaces placed between the appropriate amino acids, has a similarity included within the values indicated above. Preferably, the homology extends to the peptides comprising from 7 to 12 amino acids, even more preferably from 8 to 11 amino acids.

The expression “amino acids of the same class” is intended to mean an amino acid having substantially identical chemical properties.

In particular, this term is intended to mean amino acids having substantially the same charge and/or the same size and/or the same hydrophilicity or hydrophobicity and/or the same aromaticity.

Such combinations of amino acids include generally:

(i) glycine, alanine, valine,

(ii) isoleucine, leucine,

(iii) tryptophan, tyrosine,

(iv) aspartic acid, glutamic acid,

(v) arginine, lysine, histidine,

(vi) asparagine, glutamine,

(vii) serine, threonine,

(viii) proline, phenylalanine.

Other amino acid substitutions can be envisioned for the purpose of the present invention, in which an amino acid is replaced with another amino acid of the same class, i.e. having comparable properties, the substituting amino acid being an unnatural amino acid.

This is the case, for example, of the enantiomers and diastereoisomers of natural amino acids, of hydroxyproline, of norleucine, of ornithine, of citrulline, of cyclohexylalanine, and of Ω-amino acids such as 3-propionic acid and 4-butyric acid. Such amino acids are well known to those skilled in the art and can be prepared by known methods. Also included in the present invention are the substitutions of one or more amino acids with an unnatural amino acid carrying a, prepared by known methods. Also included in the present invention are the substitutions of one or more amino acids with an unnatural amino acid carrying a group for detecting the peptide, such as an amino acid carrying a fluorescent group or a radiolabeled amino acid.

A particular example of a homolog consists of the functional fragments of the peptides according to the invention: these are peptides corresponding to one of the sequences according to the invention in which one or more amino acids have been removed from the sequence, which conserve a bactericidal activity comparable to that described for MGD1, in particular a minimum inhibitory concentration of less than 75 μm with respect to at least one bacterial microorganism. The preferred functional fragments of the peptides according to the invention are those for which at most two amino acids are removed; even more preferably, those for which one amino acid is removed.

Usually, substitutions of amino acids of the same class allow the peptide to conserve its anti-microbial activity. Suitable substitutions may be determined by testing the anti-microbial properties of the peptides obtained, using activity assays such as those described hereinafter. The invention relates more specifically to molecules having a minimum inhibitory concentration of less than 75 μm with respect to at least one bacterial microorganism.

In particular, in the present invention, the substitution of the histidine and of the leucine of SEQ ID NO:1 with two lysines made it possible to obtain a peptide of sequence SEQ ID NO:2 comprising a disulfide bridge between the two cysteines, this cyclic peptide having an antibacterial potency greater than that of the cyclic peptide of sequence SEQ ID NO:1.

| | |
|---|---|
| SEQ ID NO:2: | C^{(S)}GGWKRKRC^{(S)} |

A subject of the invention is therefore also a peptide of sequence SEQ ID NO:2, in which the two cysteine residues are involved in a disulfide bridge, and also the pharmaceutically acceptable salts thereof, the functional fragments of this peptide, the homologous peptides and the chemical analogs of this peptide, and certain chemical derivatives of this peptide.

In particular, the invention extends to the peptides in which the amino acid sequence exhibits at least 60% similarity with a peptide corresponding to the sequence SEQ ID NO:2, even more preferably 70%, yet even more preferably 80%, preferably at least 90%, and even more favorably at least 95%, or better still 98%, similarity with the sequence SEQ ID NO:2, said sequence comprising a cysteine residue at each of its ends, these terminal cysteine residues being linked via a disulfide bridge.

More generally, a subject of the invention is peptides corresponding to formula (I) below:
C^{(S)}—X₁—X₂—X₃—X₄—X₅—X₆—X₇—C^{(S)} (I)
in which

the two cysteine residues are linked via a disulfide bridge, represented by the symbol C^{(S)},

X₁ and X₂, which may be identical or different, represent an amino acid chosen from glycine, valine, alanine and lysine,

X₃ represents an amino acid chosen from tryptophan and tyrosine,

X₄, X₅ and X₇, which may be identical or different, represent an amino acid chosen from histidine, arginine and lysine,

X₆ represents an amino acid chosen from leucine, isoleucine and lysine.

A subject of the invention is also the derivatives of the peptides of formula (I) chosen from the pharmaceutically acceptable salts thereof, the functional fragments of these peptides, the homologous peptides and the chemical analogs of these peptides and certain chemical derivatives of these peptides.

Preferably, according to the invention, at least one of the independent conditions below is satisfied:

X₁ represents glycine or lysine,

X₂ represents glycine or lysine,

X₃ represents tryptophan,

X₄ represents histidine or lysine,

X₅ represents arginine,

X₆ represents leucine or lysine,

X₇ represents arginine.

Preferably, several of the above conditions are satisfied.

The subject of the invention is also peptides comprising a sequence according to formula (I), in particular the sequence SEQ ID NO:1 or the sequence SEQ ID NO:2, in which the two cysteine residues are linked via a disulfide bridge, or peptides comprising a functional fragment of these peptides, a homolog, a chemical analog of these peptides or a chemical derivative of these peptides. In fact, one of the terminal amino acids, preferably one of the terminal cysteines, of the peptide according to formula (I) comprises a free terminal amine function, and the other comprises a free terminal carboxyl function, the two functions each being capable of being involved in a peptide bond with the C-terminal acid, respectively the N-terminal amine, of another peptide fragment.

In particular, a subject of the invention is peptides derived from MGD 1, comprising the sequence according to formula (I) in which the two cysteine residues are linked via a disulfide bridge.

The invention relates more particularly to peptides corresponding to the sequence SEQ ID NO:3, in which the two cysteine residues at 25 and 33 are linked via a disulfide bridge, the pharmaceutically acceptable salts of these peptides according to the invention, the functional fragments of these peptides, the homologous sequences, the chemical analogs of these peptides and certain chemical derivatives of these peptides.

SEQ ID NO: 3:
GFGCPNNYQCHRHCKSIPGRC^{(S)}GGYCGGWHRLRC^{(S)}TCYRCG

The invention therefore also extends to the peptides exhibiting at least 60%, preferably 70%, even more preferably 80%, preferably at least 90% and even more favorably at least 95%, or better still at least 98%, homology with the sequence SEQ ID NO:3, and in which the two cysteines at 25 and 33 form a disulfide bridge.

The peptides in question preferably comprise from 15 to 70 amino acids, even more preferably from 15 to 55 amino acids, and advantageously from 15 to 50 amino acids.

In particular, a subject of the invention is the peptides corresponding to formula (II) below:
Y₁—C^{(S)}—X₁—X₂—X₃—X₄—X₅—X₆—X₇—C^{(S)}—Y₂ (II)

in which Y₁ and Y₂ represent a peptide fragment comprising from 1 to 60 amino acids, preferably from 1 to 40 amino acids, and even more preferably from 1 to 15 amino acids; X₁, X₂, X₃, X₄, X₅, X₆ and X₇ have the same definition as above, the pharmaceutically acceptable salts of these peptides, the functional fragments of these peptides, the homologous sequences, the chemical analogs of these peptides and certain chemical derivatives of these peptides.

Preferably, Y₁ and, respectively, Y₂ have at least 60%, preferably 70%, even more preferably 80%, preferably 90% and even more preferably 95%, or better still 98%, homology with the N-terminal, respectively C-terminal, fragment of MGD 1.

According to the invention, the term “salts” relates both to the amine salts of a carboxyl function of the peptide chain and to the acid addition salts with an amine group of the same polypeptide chain. The salts of a carboxyl function can be formed with an organic or inorganic base. The inorganic salts include, for example, alkali metal salts such as sodium, potassium or lithium salts; alkaline-earth metal salts, such as, for example, calcium, barium or magnesium salts; ammonium salts, ferrous or ferric salts, and zinc, manganese, aluminum or magnesium salts. The salts with organic amines include those formed, for example, with trimethylamine, triethylamine, tri(n-propyl)amine, dicyclohexylamine, triethanolamine, arginine, lysine, histidine, ethylenediamine, glucosamine, methylglucamine, purines, piperazines, piperidines, caffeine or procaine.

The acid addition salts include, for example, the salts with inorganic acids, such as, for example, hydrochloric acid, hydrobromic acid, sulfuric acid, phosphoric acid or nitric acid; the salts with organic acids such as, for example, acetic acid, oxalic acid, tartaric acid, succinic acid, maleic acid, fumaric acid, gluconic acid, citric acid, malic acid, ascorbic acid or benzoic acid.

Chemical derivatives of the peptides according to the invention include the compounds of the alpha-amino peptides, the N-alpha acyl substituted derivatives of the form RCO—, in which R represents a linear, branched or cyclic alkyl, alkenyl, alkynyl, aryl or aralkyl group comprising from 1 to 50, preferably from 1 to 8, carbon atoms. The preferred N-alpha acyl group is the acetyl group. Such amino-terminal substituents can increase the activity of the peptide by slowing down or preventing enzymatic degradation of the peptides in vivo.

Other chemical derivatives of the peptides according to the invention include the derivatives substituted on the C-terminal acid function with a group chosen from —NH₂, and alkyloxy, alkylthio or alkylamino of the form —OR, —SR or —NHR, in which R represents a group chosen so as to facilitate the penetration in vivo of the peptides. For example, R may represent an alkyl, alkenyl, alkynyl or aryl chain or an aralkyl group, that is linear, branched or cyclic, comprising from 1 to 50, preferably from 8 to 50, carbon atoms. Preferably, the peptides according to the invention are amidated on their C-terminal function with —NH₂.

Another type of chemical derivative of the peptides according to the invention includes the derivatives carrying a pharmacophore substituent, such as a fluorescent group, a photoactivatable group, a radiolabeled group or any other group for detection by spectroscopy and quantitative evaluation of the peptide according to the invention in a biological sample without degradation of the biological sample.

Preferably, according to the invention, the amino acids of which the peptides are formed are L enantiomers. However, one or more amino acids of the peptide sequence may be replaced with its D enantiomer.

By way of the chemical analogs, in the peptides according to the invention, one or more amide peptide bonds (—CO—NH—) may be replaced with an isosteric bond such as: —CH₂NH—, —CH₂S—, —CH₂CH₂—, —CH═CH— (cis and trans), —COCH₂—, —CH(OH)CH₂— and —CH₂SO—. This substitution can be carried out by methods well known to those skilled in the art (reference may be made, for example, to: SPATOLA, Vega Data, Vol. 1, issue 3 (1983); SPATOLA, Chemistry and Biochemistry of Amino Acids Peptides and Proteins, Weinstein, ed., Marcel Dekker, New York, p. 267 (1983), MORLEY. J.-S., Trends Pharm. Sci., 463-468 (1980); HUDSON et al, Int. J. Pept. Prot. Res. 14, 177-185 (1979); SPATOLA et al, Life Sci., 38, 1243-1249 (1986); Hann, J. Chem. Soc. Perkin Trans.I 307-314 (1982); ALMQUIST et al, J. Med. Chem., 23, 1392-1398 (1980); JENNINGS-WHITE et al, EP-45665; HOLLADAY et al, Tetrahedron Lett. 24, 4401-4404 (1983). HRUBY et al, Life Sci. 31, 189-199 (1982)).

In addition, included in the present invention, by way of chemical analogs, are the peptides derived from the peptides according to the invention, in which the two cysteine residues forming the disulfide bridge have been replaced with two tryptophan residues forming a ditryptophan bridge, or with a lanthionine residue forming a monosulfide bridge, or with two amino acids, one carrying a free acid function (for example a glutamic acid) and the other carrying a free amine function (for example a lysine), both involved in a lactam bridge. According to a variant of the invention, it is also possible to envision the N-terminal and C-terminal ends of the peptide being involved in an amide bond so as to cyclize this peptide.

The peptides which are subjects of the present invention can be readily obtained by any means known to those skilled in the art, in particular by synthetic process. The means used to obtain the peptides which are subjects of the invention will be illustrated in the examples.

A subject of the invention is also the linear peptides that are precursors of the peptides with cyclic fragments according to the invention.

In particular, a subject of the invention is the linear peptides in which the sequence corresponds to formula (I) or to formula (II), and more particularly the peptides of sequence SEQ ID NO:1 and SEQ ID NO:2, these linear peptides being precursors of the cyclized peptides comprising the disulfide bridge between the end cysteines of SEQ ID NO:1 or SEQ ID NO:2. A subject of the invention is also the linear sequences that are precursors of the functional fragments of the peptides according to the invention, of the homologous sequences, of the chemical analogs of the peptides according to the invention and of certain chemical derivatives of these peptides, as defined above.

A subject of the invention is also the linear peptides for which the sequences numbered 4, 5, 6, 7, 19, 20, 21 and 22 are given in the examples.

The invention extends to the linear peptides comprising up to 30 amino acids, that are homologs of SEQ ID NO:1 or of SEQ ID NO:2, preferably to the peptides comprising from 6 to 20 amino acids, even more preferably from 6 to 15, yet more preferably from 7 to 12 amino acids.

In particular, the invention extends to the linear peptides in which the amino acid sequence exhibits at least 60% similarity with a peptide corresponding to the sequence SEQ ID NO:1 or to the sequence SEQ ID NO:2, even more preferably 70%, yet even more preferably 80%, preferably at least 90%, and even more favorably at least 95%, or better still 98%, similarity with the sequence SEQ ID NO:1 or the sequence SEQ ID NO:2.

A linear peptide that is a precursor of the peptides with a cyclic fragment according to the invention can be obtained by synthetic process or by expression in a cell expressing MGD1, isolation and then modification: cleavage of certain peptide bonds and isolation of the fragments comprising SEQ ID NO:1.

It can also be expressed by a recombinant nucleic acid molecule.

The linear peptide thus obtained by methods well known to those skilled in the art is then treated under suitable oxidation conditions so as to form the disulfide bridge between the two cysteine residues of SEQ ID NO:1 or of its homolog. When the bridge is of a type other than a disulfide bridge, those skilled in the art will be able to adapt the cyclization method to the compound in question.

The linear peptides that are precursors of the peptides with a cyclic fragment according to the invention can be obtained by expression from a nucleic acid molecule encoding the corresponding peptide. A subject of the invention is therefore also the nucleic acid molecules encoding a peptide of formula (I) or of formula (II), in particular the nucleic acid molecules encoding a peptide of sequence SEQ ID NO:1 and its homologs, and those encoding the peptide of sequence SEQ ID NO:2 and its homologs.

A nucleic acid molecule encoding a linear peptide according to the invention can be obtained either by the process of chemical synthesis or by cloning using a cell containing an MGD 1 gene or a cell encoding a cDNA corresponding to the MGD 1 mRNA.

A nucleic acid molecule according to the invention, encoding a precursor of the peptides with a cyclic fragment according to the invention, can be prepared by chemical synthesis using, as a basis, the amino acid sequence of these peptides and the knowledge of those skilled in the art with regard to the codons corresponding to each amino acid molecule.

A nucleic acid molecule encoding a precursor of the peptides with a cyclic fragment according to the invention can be cloned into a suitable vector, in particular into an expression vector, and the corresponding linear peptide can be expressed in a host cell in vitro. By these means, it is possible to obtain large amounts of the precursors of a peptide with a cyclic fragment according to the invention.

A subject of the invention is therefore also vectors containing a nucleic acid molecule encoding a precursor of the peptides with a cyclic fragment according to the invention, and host cells comprising these vectors.

The vectors and the host cells which can be used in the present invention are well known to those skilled in the art and are commercially available.

The peptides with a cyclic fragment according to the present invention represent a broad-spectrum anti-microbial activity. They are capable of reducing or inhibiting the survival or the proliferative capacity of many microorganisms: bacteria, fungi, viruses, protozoa. In particular, among the bacteria, mention may be made of Escherichia, Salmonella, Staphylococcus and Listeria. Among the fungi: Cryptococcus, Candida and Fusarium oxysporum.

The anti-microbial activity can manifest itself in the form of two distinct actions: microbicidal (bactericidal, fungicidal, virucidal or parasiticidal) inhibition, which consists in destroying or irreversibly damaging the target microorganism; microbiostatic (bacteriostatic, virostatic, fungistatic or parasitostatic) inhibition, which consists in reducing or inhibiting the proliferative capacities of the microorganism without necessarily destroying it.

The peptides with a cyclic fragment according to the invention possessing anti-microbial activity make it possible to reduce or inhibit microbial proliferation or destroy the microorganisms in a given environment. The term “microorganism” is intended to mean in particular: viruses, bacteria, fungi, protozoa, helminths and other parasites. The expression “environment which can be treated with the peptides according to the invention” denotes in particular: tissues and body fluids from humans and from animals; liquids such as water or aqueous solutions, for instance disinfectant solutions, solutions for contact lenses, solutions for washing the skin and the mucous membranes, solutions for washing the eyes; articles for hygiene; food substances; objects such as, for example, food utensils, surgical instruments; gases such as, for example, anesthetic gases used in surgery; a space, such as the walls or the floors of public or private spaces, for instance a hospital, a school, a retirement home.

A method of treatment according to the invention consists in treating an environment, excluding the human or animal body, with an effective amount of a peptide with a cyclic fragment according to the invention, so as to reduce or inhibit the ability of the microorganisms to proliferate or to survive.

The peptides according to the invention can be used as preserving agents in the food industry, the cosmetics industry, the hygiene products industry or any other industry using preserving agents, or as a disinfecting agent.

A subject of the invention is also a disinfectant composition comprising a peptide with a cyclic fragment according to the invention, in a support suitable for its purpose.

The peptides with a cyclic fragment according to the invention can also be used as a medicinal product. They can be used for preparing a therapeutic composition intended for the treatment of infections with microorganisms, in particular against bacteria and fungi.

In this case, they are combined with a pharmaceutically acceptable support. The choice of the support and of the adjuvants will be guided by the mode of administration, which will be adjusted according to the type of infection to be treated and to its location.

A subject of the invention is also a pharmaceutical composition comprising a peptide with a cyclic fragment according to the invention, in a vehicle suitable for its administration to humans or to animals.

Food products can, in order to improve the conservation thereof or to eliminate or prevent the risk of infection with microorganisms, be treated with peptides with a cyclic fragment according to the invention.

Certain food products, such as seafood and poultry, often harbor pathogenic microorganisms endemically. Fruit, vegetables and grains can be treated in order to prevent them rotting after harvesting.

The amount of peptide with a cyclic fragment to be administered to humans, or to animals, or to any environment which can be treated, depends on the activity specific to this peptide, which activity can be measured by means which will be disclosed in the examples. It also depends on the degree of the infection to be treated.

The peptide with a cyclic fragment according to the invention can be administered alone or in combination with other anti-microbial agents.

The examples below will provide a better understanding of the invention.

### EXAMPLES

I—Experimental Protocols

A—Peptide Synthesis

The synthesis was carried out by Fmoc chemistry on an Abimed AMS 422 synthesizer. The peptides were deprotected and released from the resin by treatment with trifluoroacetic acid (TFA). They were then lyophilized and their purity was estimated by HPLC on a C₁₈ column (Waters) with elution in water-acetonitrile in the presence of 0.1% of TFA and detection at 260 to 220 nm. Their molecular mass was determined by Maldi-Tof (Matrix assisted laser desorption ionization-time of flight) mass spectrometry on a Voyager Elite ED-RP device, DHB matrix. The formation of the disulfide bridge(s) took place in a 20% solution of DMSO in ammonium acetate buffer (50 mM, pH 7.5) with gentle stirring for 48 h at ambient temperature. The oxidation state was verified by mass spectrometry as above.

B—Anti-Bacterial Activity Assays

The activity of the synthetic peptides was tested with respect to the Gram+ bacteria Mycrocossus luteus, Staphylococcus aureus, Staphylococcus epidermidis and Bacillus megaterium. The activities were determined according to the protocol described by HANCOCK et al. (http://www.interchg.ubc.ca/bobh/methods.htm)

Minimum Inhibitory Concentration (MIC)

The MIC was determined for each peptide by inhibition of growth in liquid medium. Doubling dilution of peptide was realized in the wells of a Microtest plate in a volume of 10 μl. The bacteria were grown in MUELLER HINTON BROTH (MHB, Difco) liquid medium. During the exponential growth phase, a solution with an optical density of 0.001 at 600 nm was produced, and 100 μl of this suspension were added to each well. The incubation lasted 24 h at 30° C., and the optical densities of each well were then measured at 600 nm. The value of the MIC corresponds to the first well for which the optical density had not changed.

Minimum Bactericidal Concentration (MBC)

The MBC was determined in Petri dishes containing MUELLER HINTON AGAR (MHA, Difco) solid medium by plating out the content of the wells corresponding to the first three dilutions for which no bacterial growth had been observed after incubation for 24 h. The dishes were incubated for 24 h at 30° C. The value of the MBC corresponds to the lowest concentration of the peptide for which no colony developed.

C—Anti-Fungal Activity Assay

The anti-fungal activity was determined by calculating the MIC in a growth inhibition assay for the shrimp parasite fungus Fusarium oxysporum, in liquid phase, according to the protocol described by FELHBAM et al. (J. Biol. Chem., 1994, 269:33159-63). Doubling dilutions of each peptide were realized in the wells of a Microtest plate in a volume of 10 μl. 90 μl of spores resuspended in potato dextrose broth medium (DIFCO) at a final concentration of 10⁴ spores/ml were added to each well. The growth inhibition was measured after incubation for 24 h at 25° C. in the dark, by observation under an optical microscope and measurement of the increase in OD at 600 nm. The value of the MIC corresponds to the first well for which the optical density had not increased.

The Defensin MGD1:

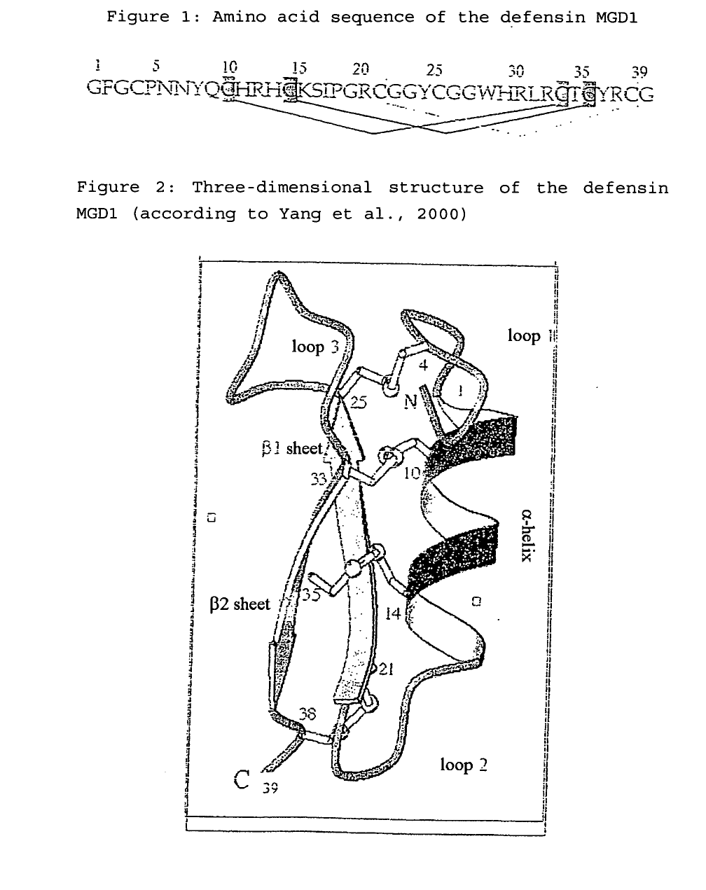

It is a small cationic protein of 4 kDa comprising 39 amino acids, isolated from the plasma and from the hemocytes of the mussel Mytilus galloprovincialis (Hubert et al. 1996, Eur. J. Biochem. 240; 302-6; Charlet et al., 1996, J. Biol. Chem. 271; 21808-13; Mitta et al., 1999, J. Cell. Sci, 112; 4233-42). The MGD1 sequence is illustrated in FIG. 1. The three-dimensional structure of the molecule, established by proton magnetic resonance (Yang et al. 2000 Biochemistry, 39:14436-47) illustrated in FIG. 2, consists of an α-helix and 2 antiparallel β-sheets, giving it a stabilized structure commonly referred to as Cs (αβ), also observed in scorpion toxins. The molecule exhibits a distribution of positive charges located over 3 domains and a hydrophobic side chain. This peptide has anti-bacterial activity against Gram+ bacteria and anti-fungal activity.

Seventeen peptides corresponding to fragments of the MDG 1 sequence were synthesized according to the methods well known to those skilled in the art, disclosed above. These peptides were tested for their anti-microbial activity and their anti-fungal activity. The results of these assays are disclosed in tables I and II.

Using the 3-D structure of the native molecule as a basis, said molecule was fractionated into 17 elements (table I). All these fragments were synthesized and their activities were assayed using antibiotic tests on Gram+ and Gram− bacteria and on a filamentous fungus. The minimum inhibitory concentration (MIC) and minimum bacteriostatic concentration (MBC) of each peptide were thus determined and were compared with those of the native molecule. From these comparisons, it emerges that:

The N-terminal portion and that corresponding to the α-helix do not have any activity: sequences 8 to 13.

The sequences 14 and 15 show no activity.

The elements corresponding to the β-sheets (devoid of the loop) do not comprise any intrinsic activity: sequence 16.

The peptide of sequence SEQ ID NO:1 corresponding to the amino acid sequence of the natural loop of the defensin MGD1, restricted by means of an unnatural disulfide bridge produced at 25-33, has anti-bacterial activity (in the MGD1 molecule, the cysteine 25 is linked to the cysteine 4 and the cysteine 33 is linked to the cysteine 10). This result is confirmed by the fact that the absence of this disulfide bridge on an analogous molecule (sequence 17) in which the two cysteines have been substituted with serine (S) gives a molecule devoid of activity. The activity of the unnatural loop is increased by the addition of the sequences of the β-sheets or of the parts of the sheets β1 or β2: peptides represented by the sequences 4 to 7:

| | |
|---|---|
| SEQ ID NO:4 | SGGYC^{(S)}GGWHRLRS^{(S)} |
| SEQ ID NO:5 | C^{(S)}GGYSGGWHRLRSTSYRC^{(S)}G |
| SEQ ID NO:6 | SGGYC^{(S)}GGWHRLRC^{(S)}TSYRSG |
| SEQ ID NO:7 | C^{(S2)}GGYC^{(S1)}GGWHRLRC^{(S1)}TSYRC^{(S2)}G |

Substitution of the histidine and of the leucine with 2 lysines (K) increases the activity (SEQ ID NO:2).

The results of these assays are surprising since:

The fact that neither the α-helix alone nor the N-terminal portion combined with the α-helix exhibits any activity is unexpected since it differs from that which is obtained with certain categories of peptides having anti-microbial activity and for which the α-helix and/or the N-terminal portion is involved in the activity. Reference may be made, for example, to: Maloy and Kari, 1995, Biopolymers 37:105-22; Juvvadi et al, 1996, J. Amer. Chem. Soc., 118: 8989-97, U.S. Pat. No. 5,821,224.

The measured biological activity of the active molecule relates to Gram+ bacteria (Micrococcus luteus, Staphylococcus aureus, Bacillus megaterium and Bacillus epidermidis) and to the filamentous fungus (Fusarium oxysporum) (table 2). This activity is located on the loop formed by amino acids 25-33 of primary structure (CGGWHRLRC), on condition that an unnatural disulfide bridge is produced between amino acids 25 and 33. The anti-bacterial potency of this structure is increased by reinforcing its cationic nature by means of substitution of the histidine 29 and of the leucine 31 with two lysines. Associating the β1 or β2 strands with a disulfide bridge at 25-33 or the β-sheet with an additional natural disulfide bridge at 21-38 confers on the molecule of sequence C^{(S2)}GGYC^{(S1)}GGWKRKRC^{(S1)}TSYRC^{(S2)}G (sequence 7) a bicyclic structure which is more active than the minimal structure and brings about the ability to lyse cells.

Table II gives the results of MIC and MBC activity (in μM) of the peptide fragments derived from the defensin MGD1 on some bacterial (Gram+) strains and on a filamentous fungus.

TABLE 1

| | | |
|---|---|---|
| Structural | | |
| elements | MIC | MBC |

| | | | | |
|---|---|---|---|---|
| | | | represented | on M leuteus |
| Peptides | AA* | Sequence | in the peptide | (μM) |

| | | | | | |
|---|---|---|---|---|---|
| SEQ ID NO:1 | 25 to 33 | CGGWHRLRC | β₁β₂ loop, | 33 | — |
| | | | cyclic | | |
| SEQ ID NO:2 | 25 to 33 | CGGWKRKRC | β₁β₂ loop, | 13 | — |
| | | | cyclic | | |
| SEQ ID NO:3 | 1 to 39 | GFGCPNNYQCHRHCKSIPGR | 1 α-helix + | — | 2 |
| (MGD 1) | | CGGYCGGWHRLRCTCYRCG | 2 β-sheets + | | |
| | | | 3 loops | | |
| SEQ ID NO:4 | 21 to 33 | SGGYCGGWHRLRC | β₁, strand, | 67 | — |
| | | | β₁β₂ loop | | |
| SEQ ID NO:5 | 21 to 39 | CGGYSGGWHRLRSTSYRCG | β-sheet, cyclic | | 13 |
| | | | at 21-38 | | |
| SEQ ID NO:6 | 21 to 39 | SGGYCGGWHRLRCTSYRSG | β3-sheet, cyclic | 32 | — |
| | | | at 25-33 | | |
| SEQ ID NO:7 | 21 to 39 | CGGYCGGWHRLRCTSYRCG | β3-sheet, cyclic | 10 | 7 |
| | | | with 2S-S | | |
| | | | bridges (25-33 | | |
| | | | and 21-38) | | |
| SEQ ID NO:8 | 1 to 10 | GFGSPNNYQS | N-terminal | >75 | — |
| | | | portion | | |
| SEQ ID NO:9 | 1 to 20 | GFGSPNNYQSHRHSKSIPGR | N-terminal, | >75 | — |
| | | | α-helix | | |
| SEQ ID NO:10 | 5 to 22 | PNNYQSHRHSKSIPGRGR | α-helix, α-β | >75 | — |
| | | | loop + arginine | | |
| SEQ ID NO:11 | 5 to 21 | PNNYQSHRHCKSIPGRC | α-helix, α-β | >75 | — |
| | | | loop, cyclic | | |
| SEQ ID NO:12 | 5 to 21 | PNNYQSHRHSKSIPGRS | α-helix, | >75 | — |
| | | | α-βloop | | |
| SEQ ID NO:13 | 7 to 16 | NYQSHRHSKS | α-helix | >75 | — |
| SEQ ID NO:14 | 14 to 22 | SKSIPGRSG | α-βloop | >75 | — |
| SEQ ID NO:15 | 14 to 22 | CKSIPGRCG | α-βloop, | >75 | — |
| | | | cyclic | | |
| SEQ ID NO:16 | 21 to 25 | CGGYSSTSYRCG | β1 and β2 | >75 | — |
| | 33 to 39 | | strands, cyclic | | |
| SEQ ID NO:17 | 25 to 33 | SGGWHRLRS | β₁β₂ loop | >75 | — |
| SEQ ID NO:18 | 30 to 39 | RLRSTSYRSG | β₂ strand | >75 | — |

*position of the amino acids in the MGD1 sequence

TABLE II

| | Micrococcus | | Staphylococcus | | Staphylococcus | | Bacillus | | Fusarium |
|---|---|---|---|---|---|---|---|---|---|
| | luteus | | aureus | | epidermidis | | megaterium | | oxysporum |
| | MIC | MBC | MIC | MBC | MIC | MBC | MIC | MBC | MIC |
| Seq No. 1 | 24 | 50 | 25 | 75 | 17 | 50 | 26 | 50 | 28 |
| Seq No. 2 | 14 | >75 | 15 | 33 | 20 | >75 | 24 | >75 | 43 |
| Seq No. 3 | 0.6 | 1.5 | 0.6 | 1.2 | 3.1 | 6.2 | 0.8 | 1.5 | 5 |
| Seq No. 4 | 26 | 13 | 67 | >75 | 38 | >75 | 52 | >75 | 41 |
| Seq No. 5 | 18 | 18 | 14 | >75 | 35 | >75 | 24 | 75 | 18.5 |
| Seq No. 6 | 29 | >75 | 19 | 75 | 24 | >75 | 25 | >75 | 14 |
| Seq No. 7 | 12 | 75 | 18 | 50 | 35 | >75 | 14 | >75 | 17 |

Assays were undertaken using the sequences SEQ ID NO:1 and SEQ ID NO:2 in order to determine the impact on the biological activity of these peptides of substituting an amino acid with another amino acid, of the same class or of a different class, and of decreasing the size of these peptides.

These sequences were numbered 19 to 24. Their sequence and their biological activity are described in table III.

It is observed that sequences 19 to 23 possess advantageous activity, although this is weaker for sequence 23. On the other hand, sequence No. 24 does not exhibit any significant biological activity.

Sequences 19 to 23 constitute another subject of the invention.

| | |
|---|---|
| SEQ ID NO:19 | C^{(S)}GGWKRLRC^{(S)} |
| SEQ ID NO:20 | C^{(S)}GGWKRKRC^{(S)} |
| SEQ ID NO:21 | C^{(S)}KKWKRKRC^{(S)} |
| SEQ ID NO:22 | C^{(S)}KWKRKRC^{(S)} |
| SEQ ID NO:23 | C^{(S)}WKRKRC^{(S)} |
| SEQ ID NO:24 | C^{(S)}WKRKC^{(S)} |

TABLE III
(μM)

| | | | | | |
|---|---|---|---|---|---|
| | Staphylo- | Staphylo- | | | |
| Micrococcus | coccus | coccus | Bacillus | Candida | Fusarium |

| | | | | | |
|---|---|---|---|---|---|
| lysodeikticus | aureus | epidermidis | megaterium | albicans | oxysporum |

| | | | | | | | | | | | | |
|---|---|---|---|---|---|---|---|---|---|---|---|---|
| Peptide | Sequences | Charges | MIC | MBC | MIC | MBC | MIC | MBC | MIC | MBC | MIC | MIC |

| | | | | | | | | | | |
|---|---|---|---|---|---|---|---|---|---|---|
| Seq No. 19 | CGGWKRLRC | +3 | 13 | | 33 | 20 | | 20 | 52 | 36 |
| Seq No. 20 | CGGWKRKRC | +4 | 8 | | 23 | 16 | | 39 | 16 | 46 |
| Seq No. 21 | CKKWKRKRC | +6 | 6 | 9.9 | 4.4 | 9.9 | 6.8 | | 14.8 | 22.2 |
| Seq No. 22 | CKWKRKRC | +5 | 14.8 | 14.8 | 6.6 | 14.8 | | | 14.8 | 33.3 |
| Seq No. 23 | CWKRKRC | +4 | 22.2 | | 22.2 | 22.2 | | | 33.3 | >50 |
| Seq No. 24 | CWKRKC | +3 | >50 | | >50 | >50 | | | >50 | |

1. A compound chosen from:
the peptides corresponding to formula (I):
C^{(S)}—X₁—X₂—X₃—X₄—X₅—X₆—X₇—C^{(S)} (I)
in which
the two cysteine residues are linked via a disulfide bridge, represented by the symbol C^{(S)},
X₁ and X₂, which may be identical or different, represent an amino acid chosen from glycine, valine, alanine and lysine,
X₃ represents an amino acid chosen from tryptophan and tyrosine,
X₄, X₅ and X₇, which may be identical or different, represent an amino acid chosen from histidine, arginine and lysine,
X₆ represents an amino acid chosen from leucine, isoleucine and lysine, and also:
the derivatives of these peptides selected from:
the pharmaceutically acceptable salts of these peptides,
the functional fragments of these peptides,
the chemical analogs of these peptides, chosen from those in which: one or more amino acids of the peptide sequence have been replaced with their D enantiomer; one or more amide peptide bonds (—CO—NH—) have been replaced with an isosteric bond such as: —CH₂NH—, —CH₂S—, —CH₂CH₂—, —CH═CH— (cis and trans), —COCH₂—, —CH(OH)CH₂— and —CH₂SO—; one or more amino acids have been replaced with an unnatural amino acid; the two cysteine residues forming the disulfide bridge have been replaced with two tryptophan residues forming a ditryptophan bridge or with a lanthionine residue forming a monosulfide bridge, or with two amino acids, one carrying a free acid function and the other carrying a free amine function, both involved in a lactam bridge, or a peptide in which the disulfide bridge has been replaced with an amide bond between the N-terminal and C-terminal ends of the peptide,
the chemical derivatives of this peptide, chosen from: the des-alpha-amino peptide compounds; the N-alpha acyl substituted derivatives of the form RCO—, in which R represents a linear, branched or cyclic alkyl, alkenyl, alkynyl, aryl or aralkyl group comprising from 1 to 50 carbon atoms; the derivatives substituted on the C-terminal acid function with a group chosen from —NH₂, and alkyloxy, alkylthio or alkylamino of the form —OR, —SR or —NHR, in which R represents an alkyl, alkenyl, alkynyl or aryl chain or an aralkyl group, that is linear, branched or cyclic, comprising from 1 to 50 carbon atoms, the derivatives carrying a pharmacophore substituent.

2. The cyclic peptide as claimed in claim 1, characterized in that it corresponds to the sequence SEQ ID NO:1: C^{(S)}GGWHRLRC^{(S)} in which the two cysteine residues are linked via a disulfide bridge.

3. The peptide as claimed in claim 1, characterized in that it corresponds to the sequence SEQ ID NO:2: C^{(S)}GGWKRKRC^{(S)} in which the two cysteine residues are linked via a disulfide bridge.

4. A peptide in which the amino acid sequence exhibits at least 60% similarity with the peptide corresponding to formula (I) as claimed in claim 1, said sequence comprising a cysteine residue at each of its ends, these terminal cysteine residues being linked via a disulfide bridge.

5. A peptide in which the amino acid sequence exhibits at least 60% similarity with the peptide corresponding to Xv sequence SEQ ID NO: 1 or to the sequence SEQ ID NO:2, said sequence comprising a cysteine residue at each of its ends, these terminal cysteine residues being linked via a disulfide bridge.

6. The peptide as claimed in claim 4, characterized in that it comprises from 6 to 15 amino acids.

7. The peptide as claimed in claim 6, characterized in that it comprises from 7 to 12 amino acids.

8. A compound characterized in that it is chosen from:
the peptides corresponding to formula (II):
Y₁—C^{(S)}—X₁—X₂—X₃—X₄—X₅—X₆—X₇—C^{(S)}—Y₂ (II)
in which Y₁ and Y₂ represent a peptide fragment comprising from 1 to 60 amino acids;
X₁ and X₂, which may be identical or different, represent an amino acid chosen from glycine, valine, alanine and lysine,
X₃ represents an amino acid chosen from tryptophan and tyrosine,
X₄, X₅ and X₇, which may be identical or different, represent an amino acid chosen from histidine, arginine and lysine,
X₆ represents an amino acid chosen from leucine, isoleucine and lysine, and also:
the derivatives of these peptides selected from:
the pharmaceutically acceptable salts of these peptides,
the functional fragments of these peptides,
the chemical analogs of these peptides, chosen from those in which: one or more amino acids of the peptide sequence have been replaced with their D enantiomer; one or more amide peptide bonds (—CO—NH—) have been replaced with an isosteric bond such as: —CH₂NH—, —CH₂S—, —CH₂CH₂—, —CH═CH— (cis and trans), —COCH₂—, —CH(OH)CH₂— and —CH₂SO—; one or more amino acids have been replaced with an unnatural amino acid; the two cysteine residues forming the disulfide bridge have been replaced with two tryptophan residues forming a ditryptophan bridge or with a lanthionine residue forming a monosulfide bridge, or with two amino acids, one carrying a free acid function and the other carrying a free amine function, both involved in a lactam bridge, or a peptide in which the disulfide bridge has been replaced with an amide bond between the N-terminal and C-terminal ends of the peptide,
the chemical derivatives of this peptide, chosen from: the des-alpha-amino peptide compounds; the N-alpha acyl substituted derivatives of the form RCO—, in which R represents a linear, branched or cyclic alkyl, alkenyl, alkynyl, aryl or aralkyl group comprising from 1 to 50 carbon atoms; the derivatives substituted on the C-terminal acid function with a group chosen from —NR₂, and alkyloxy, alkylthio or alkylamino of the form —OR, —SR or —NHR, in which R represents an alkyl, alkenyl, alkynyl or aryl chain or an aralkyl group, that is linear, branched or cyclic, comprising from 1 to 50 carbon atoms, the derivatives carrying a pharmacophore substituent.

9. The peptide as claimed in claim 8, characterized in that it comprises a peptide of sequence SEQ ID NO:1 in which the two cysteine residues are linked via a disulfide bridge.

10. The peptide as claimed in claim 8, characterized in that it comprises a peptide of sequence SEQ ID NO:2 in which the two cysteine residues are linked via a disulfide bridge.

11. The peptide as claimed in claim 9, characterized in that it is derived from MGD 1.

12. The peptide as claimed in claim 9, characterized in that it corresponds to the sequence SEQ ID NO:3, in which the two cysteine residues at 25 and 33 are linked via a disulfide bridge.

13. A peptide characterized in that it exhibits at least 60%, homology with the peptide corresponding to the sequence SEQ ID NO:3 as claimed in claim 12, and in which the two cysteines at 25 and 33 form a disulfide bridge.

14. The peptide as claimed in claim 13, characterized in that it comprises from 15 to 70 amino acids.

15. The peptide as claimed in claim 1, characterized in that it corresponds to one of the sequences:

| | |
|---|---|
| SEQ ID NO:4 | SGGYC^{(S)}GGWHRLRC^{(S)} |
| SEQ ID NO:5 | C^{(S)}GGYSGGWHRLRSTSYRC^{(S)}G |
| SEQ ID NO:6 | SGGYC^{(S)}GGWHRLRC^{(S)}TSYRSG |
| SEQ ID NO:7 | C^{(S2)}GGYC^{(S1)}GGWHRLRS^{(S1)}TSYRC^{(S2)}G |
| SEQ ID NO:19 | C^{(S)}GGWKRLRC^{(S)} |
| SEQ ID NO:20 | C^{(S)}GGWKRKRC^{(S)} |
| SEQ ID NO:21 | C^{(S)}KKWKRKRC^{(S)} |
| SEQ ID NO:22 | C^{(S)}KWKRKRC^{(S)} |
| SEQ ID NO:23 | C^{(S)}WKRKRC^{(S)} |

16. A linear peptide characterized in that it is chosen from those corresponding to one of the sequences:

| | |
|---|---|
| SEQ ID NO:1 | CGGWHRLRC |
| SEQ ID NO:2 | CGGWKRKRC |
| SEQ ID NO:4 | SGGYCGGWHRLRC |
| SEQ ID NO:5 | CGGYSGGWHRLRSTSYRCG |
| SEQ ID NO:6 | SGGYCGGWHRLRCTSYRSG |
| SEQ ID NO:7 | CGGYCGGWHRLRCTSYRCG |
| SEQ ID NO:19 | CGGWKRLRC |
| SEQ ID NO:20 | CGGWKRKRC |
| SEQ ID NO:21 | CKKWKRKRC |
| SEQ ID NO:22 | CKWKRKRC |
| SEQ ID NO:23 | CWKRKRC |

17. A method for preparing a peptide as claimed in claim 1, comprising a disulfide bridge, characterized in that it comprises at least a first step chosen from chemical synthesis, expression in a cell expressing MGD1, and expression by a recombinant nucleic acid molecule, and at least a second step consisting of a treatment under suitable oxidation conditions so as to form the disulfide bridge between the two cysteine residues.

18. A nucleic acid molecule encoding a peptide as claimed in claim 16.

19. A vector containing a nucleic acid molecule as claimed in claim 18.

20. A host cell comprising a vector as claimed in claim 19.

21. The use of a peptide as claimed in claim 1, for reducing or inhibiting microbial proliferation or destroying the microorganisms in an environment, excluding the human or animal body.

22. The use as claimed in claim 21, characterized in that the environment to be treated is chosen from: liquids, articles for hygiene, food substances, objects for food or surgical purposes, gases, and a space.

23. A pharmaceutical composition characterized in that it comprises at least one peptide as claimed in claim 1, in a pharmaceutically acceptable support.

24. A disinfectant composition characterized in that it comprises at least one peptide as claimed in claim 1.
